(19) United States
(12) Patent Application Publication
(10) Pub. No.: US 2024/0355458 A1
(43) Pub. Date: Oct. 24, 2024

(54) FINDING-SPECIFIC TRAINING OF MACHINE-LEARNING MODELS USING IMAGE INPAINTING

(71) Applicant: KONINKLIJKE PHILIPS N.V., EINDHOVEN (NL)

(72) Inventors: JOËL VALENTIN STADELMANN, MOSCOW (RU); HRISHIKESH NARAYANRAO DESHPANDE, HAMBURG (DE)

(21) Appl. No.: 18/684,214

(22) PCT Filed: Aug. 17, 2022
(86) PCT No.: PCT/EP2022/072937

§ 371 Date: Feb. 16, 2024

(30) Foreign Application Priority Data
Aug. 20, 2021 (RU) 2021124799

(51) Int. Cl. G16H 30/40; G06T 5/77; G06T 7/12; G06V 10/44; G06V 10/764

(52) U.S. Cl. CPC G16H 30/40; G06T 5/77; G06T 7/12; G06V 10/44; G06V 10/764; G06T 2207/10072; G06V 2201/07

(57) ABSTRACT

The present invention relates to image processing. In order to generate ground truth data for training a machine-learning model, a computer-implemented method is proposed for training a machine-learning model for classifying image features in an image acquired by a medical scanner. The method comprises: receiving an image acquired by a medical scanner, identifying an artefact in the image, segmenting the artefact to obtain a segmentation mask of a region, on which to perform inpainting, performing inpainting on the region to obtained an inpainted image, and incorporating the inpainted image into a training set for the development of the machine-learning model.

### FIELD OF THE INVENTION

The present invention relates to a computer-implemented method and an apparatus for training a machine-learning model for classifying image features in an image acquired by a medical scanner. The present invention further relates to a medical imaging system, to a computer program product, and to a computer readable medium.

### BACKGROUND OF THE INVENTION

Machine-learning algorithms, such as convolutional neural networks (CNNs), achieve state-of-the-art results in many computer vision tasks. They find applications in radiology, where the number of studies considerably surpasses the number of radiologists available to read them. However, machine-learning algorithms may rely on the co-occurrence of visual features and given labels in order to make the classification decision.

The visual features that a machine-learning algorithm associates with an intended label are not always concordant with the actual manifestation of the label on the image. In the case of the pneumothorax label, a machine-learning algorithm probably learns to associate the label with the presence of tubes used to drain the pneumothoraxes. As a result, such machine-learning algorithm might miss pneumothorax instances that are not yet treated. Thus, the initial phase of ground truth generation is most often that factor that limits the final performance of the algorithm due to the inaccuracies in the labelling due to the presence of an artefact.

### SUMMARY OF THE INVENTION

It is thus an object of the present invention to provide an improved apparatus and method for generating ground truth data for training a machine-learning model.

The object of the present invention is solved by the subject-matter of the independent claims. Further embodiments and advantages of the invention are incorporated in the dependent claims. Furthermore, it shall be noted that all embodiments of the present invention concerning a method might be carried out with the order of the steps as described, nevertheless this has not to be the only and essential order of the steps of the method as presented herein. The method disclosed herein can be carried out with another order of the disclosed steps without departing from the respective method embodiment, unless explicitly mentioned to the contrary hereinafter.

According to a first aspect of the present invention, there is provided a computer-implemented method for training a machine-learning model for classifying image features in an image acquired by a medical scanner, comprising:
a) receiving an image acquired by a medical scanner;
b) identifying an artefact in the image;
c) segmenting the artefact to obtain a segmentation mask of a region, on which to perform inpainting;
d) performing inpainting on the region to obtained an inpainted image; and
e) incorporating the inpainted image into a training set for the development of the machine-learning model.

The method proposed in the present disclosure steers the training of a machine-learning algorithm towards the manifestation of a label on an image. The proposed method removes other features that often but not always co-occur with the label. A simple removal of the co-occurring objects would result in its silhouette still being present on the image. Thus, in order to avoid the association of the label with the silhouette, the co-occurring object is replaced by the natural appearance of the tissues at the respective locations. It is achieved by inpainting, using either mathematical methods, generative models such as generative adversarial networks (GANs), variational autoencoders (VAEs), normalizing flows or other approaches. The inpainted image is then added to a training set for training a machine-learning model.

In some examples, annotated ground truth data may be generated by using a fully automated procedure to compute one or more features (e.g., landmarks, organ boundaries, region-of-interest, and/or image labels) in the inpainted image. One option is to use an image processing algorithm, such as an image detection algorithm that finds an approximate centre and bounding box of the one or more image in the inpainted image. Another option is to derive the one or more features from a configuration parameter of a scan volume that was planned by an operator.

The proposed method may have one or more of the following advantages:

The images derived in this manner without artefacts (e.g., foreign objects) may be employed for training a machine-learning model and as a consequence may prevent the machine-learning algorithm from missing pathologies or image feature that are not co-occurring with such artefacts (e.g., foreign object).

Quality assessment of e.g., chest x-rays is required for the automation of their processing. By inpainting external objects before quality assessment, one could prevent them from hindering the field of view and rotations checks, and thus improve the quality of automated chest x-ray processing.

By using all available information from previous studies (e.g., numerous chest x-rays, radiological reports, etc.), one could remove external objects that would hide significant findings. For instance, one could inpaint on the frontal view a lung nodule, which was observed on a previous lateral view, and that is hidden behind a pacemaker's electronic control board.

Further, the position and the size of an internal anatomy of interest may be automatically labelled in the inpainted image to generate annotated ground truth data. The annotated ground truth data may be generated from routine examinations at one or more clinical sites. The continuous and unsupervised process of acquiring annotated ground truth data provides a basis for the generation of a large ground truth database, thereby overcoming the disadvantage of the limited number of training sets obtained by a manual or semi-automated process. The accuracy of the labelling may be improved, as the inpainted images derived in this manner do not contain artefacts. One advantage of the proposed method is the ground truth data may be generated continuously from routine examinations and research examination in a fully automated manner, thereby further and continuously extending the training set database. The large number of annotated ground truth data may be essential for training the machine-learning model. For example, for deep learning approaches (e.g., CNNS), it is essential to train the neural networks with a very large number of data to avoid overfitting effects to ensure that the trained system well generalizes to unseen data.

According to an embodiment of the present invention, the method further comprising extracting one or more features from the inpainted image, and incorporating data that labels the one or more features in the inpainted image into the training set.

For example, annotated ground truth data may be generated by using a fully automated procedure to compute one or more features such as landmarks, organ boundaries, region-of-interest, and/or image labels in the inpainted image.

In some examples, the one or more features may be extracted from the inpainted image using an image processing algorithm.

In some examples, the one or more features may be derived from an exam metadata that comprises a configuration parameter of a scan volume that was planned by an operator.

According to an embodiment of the present invention, the artefact comprises one or more of a motion-induced artefact in the image, a text marker added onto the image, and an artefact induced by an object that is imaged along with the patient.

According to an embodiment of the present invention, the image comprises one or more of the following images: chest x-ray, computed tomography, magnetic resonance, ultrasound, positron emission tomography, and single photon emission computed tomography image.

In some examples, the image may be a medical image captured by a smartphone.

In some examples, the image is a localizer or surview image.

According to an embodiment of the present invention, step b) further comprises:
applying the machine-learning model to classify image features in the image;
analysing a classification result to determine whether a particular class exhibits an error exceeding a threshold; and
if a particular class exhibits an error exceeding a threshold, determining a co-occurrence of an artefact in the image.

Stated differently, if a particular class exhibits a significant error, it could be the sign of a label's manifestation co-occurring with a different feature. The hypothesis may be confirmed in two ways: (a) by retraining a machine-learning algorithm in a smaller context, where the co-occurring feature is absent; and (b) by observing a class activation map (CAM) in order to gain understanding of the most influential regions of the image.

Thus, the present disclosure proposes a method for the steering of the decision based on the manifestation of a label in an image, instead of the presence of externals.

According to an embodiment of the present invention, step c) comprises:
segmenting the artefact based on a threshold on a class activation map, CAM;
segmenting the artefact with a UNET-like structure: or
segmenting the artefact with a deep generative machine-learnt model.

According to an embodiment of the present invention, step d) comprises:
performing inpainting with a deep generative machine-learnt model: or
performing inpainting with a partial differential equation.

According to an embodiment of the present invention, the deep generative machine-learnt model comprises at least one of a generative adversarial network (GAN), a variational autoencoder (VAE), and normalizing flows.

According to an embodiment of the present invention, the method further comprises training the machine-learning model on the training set.

This may prevent the machine-learning algorithm from missing pathologies or image feature that are not co-occurring with such artefacts (e.g., foreign object).

According to an embodiment of the present invention, the method further comprises displaying the inpainted image.
According to a second aspect of the present invention, there is provided an apparatus for training a machine-learning model for classifying image features in an image acquired by a medical scanner. The apparatus comprises an input unit, a processing unit, and
an output unit. The input unit is configured to receive an image acquired by a medical scanner. The processing unit is configured to identify an artefact in the image, to segment the artefact to obtain a segmentation mask of a region, on which to perform inpainting, and to perform inpainting on the region to obtained an inpainted image. The output unit is configured to incorporate the inpainted image into a training set for the development of the machine-learning model.

According to an embodiment of the present invention, the processing unit is further configured to extract one or more features from the inpainted image, and to incorporate data that labels the one or more features in the inpainted image into the training set via the output unit.

According to a third aspect of the present invention, there is provided a medical imaging system. The medical system comprises a medical imaging apparatus configured to acquire an image of a patient and an apparatus according to the second aspect and any associated example.

The medical imaging apparatus may be e.g., an x-ray imaging apparatus, an MR imaging apparatus, a CT imaging apparatus, or a PET imaging apparatus. Further examples of the medical imaging apparatus may include a combined therapy/diagnostic apparatus, such as an MR-Linac apparatus, an MR proton therapy apparatus, and/or a cone beam CT apparatus.

According to a fourth aspect of the present invention, there is provided a computer program product comprising instructions which, when executed by at least one processing unit, cause the at least one processing unit to perform the steps of the method according to the first aspect and any associated example.

According to a further aspect of the present invention, there is provided a computer readable medium having stored the program product.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 shows an exemplary apparatus for training a machine-learning model for classifying image features in an image acquired by a medical scanner

FIGS. 2A and 2B show an example of the CAMs.

FIG. 3 shows an exemplary medical imaging system.

FIG. 4 shows a flow chart of a method for training a machine-learning model for classifying image features in an image acquired by a medical scanner.

### DETAILED DESCRIPTION OF EMBODIMENTS

The inventors of the present disclosure have found out that when a pathology co-occurs with an external or internal foreign object in an image, a machine-learning algorithm, such as CNNs, could possibly learn to focus on the object instead of the pathology. This may cause the machine-learning algorithm to correlate the presence of foreign object with the pathology and could lead to the misclassification of pathological cases when it does not co-occur with the foreign object. In addition, such foreign object could block the image features in the background, which might be of prime importance in machine-learning-based approaches such as pathology detection or image quality assessment.

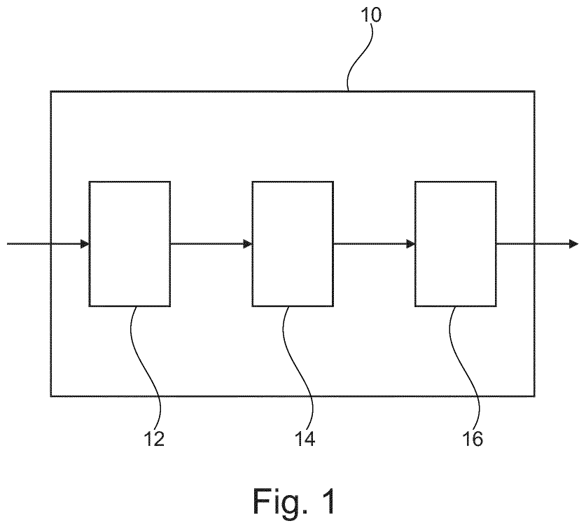

Towards this end, an apparatus is proposed for training a machine-learning model for classifying image features in an image acquired by a medical scanner. Examples of the machine-learning model may include, but are not limited to, convolutional neural networks (CNNs), decision trees, support vector machines (SVMs), and boosted classifiers. An exemplary apparatus 10 is shown in FIG. 1.

The following description focuses on embodiments of the present disclosure applicable to artefacts induced by a foreign object. However, it will be appreciated that the invention is not limited to this application but may be applied to other artefacts, such as text markers and motion-induced artefacts. For example, inpainting motion corrupted 2D slices in CT/MR images may be performed for possibly similar applications. Often, single slice in 3D image gets affected by large motion artefacts, which could degrade the automatic image classification results. If such 2D slices are corrected by similar inpainting method, it is possible to add robustness to the automatic image classification or quality assessment algorithms, in the exactly similar manner as we discuss below for foreign objects in e.g. chest x-ray images.

The input unit 12 is configured to receive an image acquired by a medical scanner. The image may comprise a two-dimensional (2D), three-dimensional (3D), or four-dimensional (4D) image, acquired by various acquisition modalities including, but not limited to, x-ray imaging, computed tomography (CT) imaging, magnetic resonance (MR) imaging, ultrasound imaging, positron emission tomography (PET) imaging, and single photon emission computed tomography (SPECT) imaging. Further examples of the medical imaging apparatus may include a combined therapy/diagnostic apparatus, such as an MR-Linac apparatus, an MR proton therapy apparatus, and/or a cone beam CT apparatus. The image may also comprise localizer images or scout images, which are used in MR and CT studies to identify the relative anatomical position of a collection of cross-sectional images. The image may include medical images captured from a smartphone.

The processing unit 14 is configured to identify an artefact in the image. Examples of the artefact may include, but are not limited to, a motion-induced artefact, a text marker added onto the image, and an artefact induced by a foreign object that is imaged along with the patient.

In an example, the artefact may result from foreign bodies external to the patient. Examples of the foreign bodies that may be imaged along the patient may include, but are not limited to, pacemakers, probes, tubes, implants, jewellery, etc. In an example, it is common to see jewellery artefacts on imaging examinations, most commonly plain radiographs, although also on other modalities, where they can produce unhelpful artefacts that may obscure important structures and preclude confident diagnostic evaluation. In a further example, the artefact may result from implanted devices. For example, external objects such as tubes or drains could often appear on images that are flagged for pneumothorax. The motion-induced artefact in the image occurs with voluntary or involuntary patient movement during image acquisition. For example, misregistration artefacts, which appear as blurring, streaking, or shading, are caused by patient movement during a CT scan. Blurring also occurs with patient movement during radiographic examinations. If patient movement is voluntary, patients may require immobilization or sedation to prevent this. Involuntary motion, such as respiration or cardiac motion, may cause artefacts that mimic pathology in surrounding structures.

The identification of artefacts may according to some examples be realized by analysing the classification results during the development of a machine-learning model. For example, the machine-learning model may be applied to classify image features in the image. The classification results may be analysed to determine whether a particular class exhibits an error exceeding a threshold. If a particular class exhibits an error exceeding a threshold, a co-occurrence of an artefact in the image can be determined. In other words, if a particular class exhibits a significant error, it could be the sign of a label's manifestation co-occurring with a different feature. This hypothesis may be confirmed in two ways. The first way is to retrain the machine-learning model (e.g., CNN) in a smaller context, where the co-occurring feature is absent. The second way is to observe CAMs in order to gain understanding of the most influential regions of the image.

Class activation mapping (CAM) is a method to generate heatmaps of images that show which areas were of high importance in terms of a neural networks for image classification. There are several variations on the method including Score-CAM and Grad-CAM (Gradient Weighted Class Activation Mapping). The heatmaps generated by CAM is a visualization that can be interpreted as telling us where in the image the neural net is (metaphorically) looking to make its decision, however they do not tell us what particularities it might be looking at. In some cases, the heatmaps from CAM can be used not only to inform what pixels were important in the neural network classification process of an image, but also for object localization.

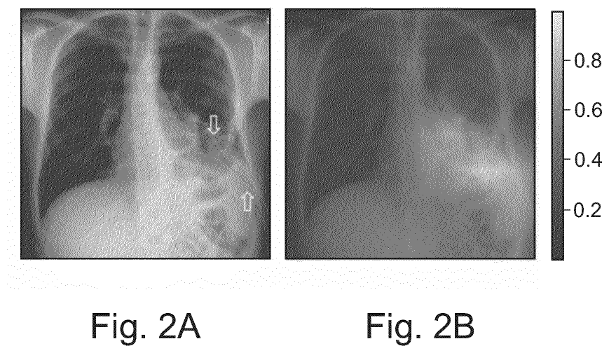

An example of the CAMs is shown in FIGS. 2A and 2B. In this example. FIG. 2A shows a chest x-ray, on which a drain is visible and heighted by arrows. The corresponding CAM is shown in FIG. 2B, which maps the relative influence of different parts of the image in classifying this chest x-ray image for pneumothorax. It could be seen that the most constituent part in the pneumothorax classification is the drain. As this particular class exhibits a significant error, it could be the sign of a label's manifestation co-occurring with an artefact, such as the drain in this example.

Turning back to FIG. 1, the processing unit 14 is further configured to segment the artefact to obtain a segmentation mask of a region, on which to perform inpainting.

The segmentation of the image may according to some examples to be realized with a UNET-like structure. This architecture contains two paths. First path is the contraction path (also called as the encoder) which is used to capture the context in the image. The encoder is just a traditional stack of convolutional and max pooling layers. The second path is the symmetric expanding path (also called as the decoder) which is used to enable precise localization using transposed convolutions. Thus, it is an end-to-end fully convolutional network (FCN), i.e, it only contains Convolutional layers and does not contain any Dense layer because of which it can accept image of any size. In this case, it may require a substantial dataset of annotated images for training.

In some examples, it is possible to use a deep generative machine-learned model that minimizes the distance between the source image, on which the external object is present, and the generated image, on which it is absent. Such deep generative machine-learned models could be trained without annotation by inserting artefact(s) in the images.

In some examples, because of the next inpainting step, segmentation may not need to be extremely precise. One could use thresholds on CAMs in order to coarsely highlight the artefact(s) and remove them.

The processing unit 14 is further configured to perform inpainting on the region to obtain an inpainted image.

Several inpainting techniques may be performed on the region to obtain an inpainted image.

In some examples, the step of inpaining is performed with a partial differential equation. A digital image is essentially a two-dimensional matrix of integers, with each integer representing the colour or grayscale value of an individual pixel. The segmentation mask in the image are represented by unknown values in the matrix. The partial differential equation thus fills in these missing values based on the values of nearby pixels. Examples of the partial differential equation may include, but are not limited to, Fourier's heat equation, the Perona-Malik equation, forth-order total variation equation, etc.

In some examples, the inpainting technique utilizes a deep generative machine-learnt model. The deep generative machine-learnt model, if trained properly, is capable of learning the latent distribution of data and using that information to create new samples. This ability of the deep generative machine-learnt model makes them perfect for image inpainting, which is filling the missing part in images with plausible pixels. Examples of the deep generative machine-learnt model may include, but are not limited to, generative adversarial networks (GANs), variational autoencoders (VAEs), and normalizing flows.

The output unit 16 is configured to incorporate the inpainted image into a training set for the development of the machine-learning model. For example, the output unit 16 is configured to be coupled to a training set database (not shown) for adding the inpainted image to the training set database for training the machine-learning model. The training set database may be stored in an internal storage device of the apparatus 10. Alternatively or additionally, the training set database may be stored in an external storage device, e.g., in a cloud storage.

In some examples, one or more features may be extracted from the inpainted image and annotated automatically. The one or more features extracted from the inpainted image may include, but are not limited to, landmarks, organ boundaries, region-of-interest, and/or image labels in the inpainted images. For example, the one or more features may be extracted from the inpainted image using an image processing algorithm, such as a detection algorithm that would find the approximate centre and bounding box of relevant features. Alternatively or additionally, the one or more features may be extracted from the inpainted image based on exam metadata that comprises a configuration parameter of a scan volume that was planned by an operator. The configuration parameter may be used to extract the one or more features from the inpainted image. Thus, in addition to the inpainted images, the one or more features extracted from the inpainted images and data that labels the one or more features may also be added to the training set for training the machine-learning model.

The apparatus is proposed to implant the artefact to improve the image classification and image quality assessment, which otherwise get degraded because of the artefact hiding the anatomical tissues underneath. Further, continuous and unsupervised acquisition of annotated ground truth data can be achieved, which provides a basis for ideal generation of a large ground truth database. As no manual search and edition of artefacts in the image is required, time efficiency may be improved. Additionally, the ground truth database may be further and continuously extended. If data with new and not foreseen characteristics arise, or if the desired outcome of the trained system needs to be adapted, the machine-learning model trained with the ground-truth database can accommodate for these changes happening over time.

The apparatus 10 may be embodied as, or in, a device or apparatus, such as a server, workstation, imaging system or mobile device. The apparatus 10 may comprise one or more microprocessors or computer processors, which execute appropriate software. The processing unit of the apparatus may be embodied by one or more of these processors. The software may have been downloaded and/or stored in a corresponding memory, e.g., a volatile memory such as RAM or a non-volatile memory such as flash. The software may comprise instructions configuring the one or more processors to perform the functions described with reference to the processor of the system. Alternatively, the functional units of the apparatus, e.g., input unit, processing unit, and output unit, may be implemented in the device or apparatus in the form of programmable logic, e.g., as a Field-Programmable Gate Array (FPGA). The input unit and the output unit may be implemented by respective interfaces of the apparatus. In general, each functional unit of the apparatus may be implemented in the form of a circuit. It is noted that the apparatus 10 may also be implemented in a distributed manner. For example, some or all units of the apparatus may be arranged as separate modules in a distributed architecture and connected in a suitable communication network, such as a 3rd Generation Partnership Project (3GPP) network, a Long Term Evolution (LTE) network. Internet. LAN (Local Area Network). Wireless LAN (Local Area Network). WAN (Wide Area Network), and the like.

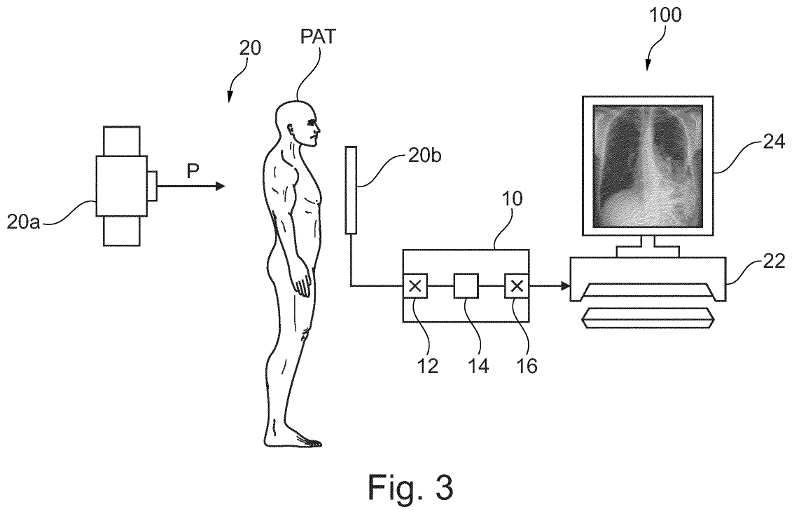

The apparatus 10 may be installed in a hospital or other environment allowing scanning under clinical-like conditions to collect and process data from a routine examination and a research examination at one or more clinical sites. FIG. 3 shows a medical imaging system 100 that may be used to acquire ground truth data or annotated ground truth data continuously from a routine examination. The medical imaging system 100 comprises a medical imaging apparatus 20 and the apparatus 10 as described above.

In the example of FIG. 3, the medical imaging system 100 is a chest radiography imaging system and the medical imaging apparatus 20 is an x-ray imaging apparatus. In some other examples (not shown), the medical imaging apparatus 10 may be e.g., an MR imaging apparatus, a CT imaging apparatus, or a PET imaging apparatus. Further examples of the medical imaging apparatus 10 may include a combined therapy/diagnostic apparatus, such as an MR-Linac apparatus, an MR proton therapy apparatus, and/or a cone beam CT apparatus.

The x-ray imaging apparatus 20 comprises an x-ray source 20a and an x-ray detector 20b. The x-ray detector 20b is spaced from the x-ray source to accommodate a patient PAT to be imaged. In general, during an image acquisition, a collimated x-ray beam (indicated with arrow P) emanates from the x-ray source 20a, passes through the patient PAT at a region of interest (ROI), experiences attenuation by interaction with matter therein, and the attenuated beam then strikes the surface of the x-ray detector 20b. The density of the organic material making up the ROI determines the level of attenuation. That is the rib cage and lung tissue in the chest radiography imaging examination. High density material (such as bone) causes higher attenuation than less dense materials (such as lung tissue). The registered digital values for the x-ray are then consolidated into an array of digital values forming an x-ray projection image for a given acquisition time and projection direction.

Overall operation of the x-ray imaging apparatus 20 may be controlled by an operator from a console 22. The console 22 may be coupled to a screen or monitor 24 on which the acquired x-ray images or imager settings may be viewed or reviewed. An operator such as a medical lab technical can control via the console 22 an image acquisition run by releasing individual x-ray exposures for example by actuating a joy stick or pedal or other suitable input means coupled to the console 22.

In the example of FIG. 3, the patient PAT stands facing a flat surface behind which is the x-ray detector 20b. According to a different example (not shown), the x-ray imaging apparatus may be of the C-arm type and the patient PAT is actually lying on an examination table instead of standing.

The apparatus 10 may be an apparatus as described with respect to FIG. 1. In the example of FIG. 3, the apparatus 10 is a separate device configured to communicate with the medical imaging apparatus 20 through a wireless and/or a wire-based interface. However, in alternative examples (not shown), the apparatus 10 may resident in the console 22 running as software routines. The apparatus 10 is configured to generate ground truth data comprising inpainted images. Optionally, the apparatus 10 may be configured to generate annotated ground truth data comprising, in addition to the inpainted images, data that labels one or more features extracted from the inpainted image.

The system 100 may comprise a storage device (not shown) for storing the training set database obtained from one or more clinical sites, e.g., in form of a cloud storage for storing the training set database 18. In other words, annotated ground truth data may be collected and processed continuously in a fully automated manner on request and upon agreement with one or more clinical sites. The annotated ground truth data may be shared and aggregated, e.g., via cloud technology. This may be done in a way which is compliant with regulation on privacy data, for example, by ensuring that the input data are anonymized and that only an intermediate layer of processed data is shared and stored, in which the anonymity is guaranteed.

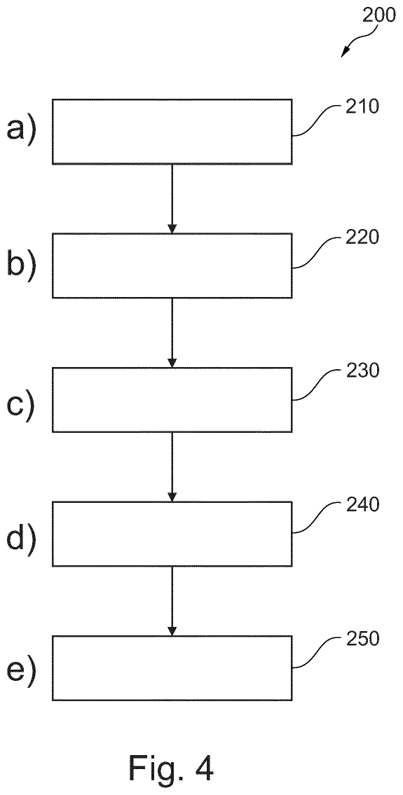

FIG. 4 shows a flow chart of a method 200 for training a machine-learning model for classifying image features in an image acquired by a medical scanner. Examples of the machine-learning model may include, but are not limited to, convolutional neural networks (CNNs), decision trees, support vector machines (SVMs), and boosted classifiers.

The computer-implemented method 200 may be implemented as a device, module, or related component in a set of logic instructions stored in a non-transitory machine- or computer-readable storage medium such as random access memory (RAM), read only memory (ROM), programmable ROM (PROM), firmware, flash memory, etc., in configurable logic such as, for example, programmable logic arrays (PLAs), field programmable gate arrays (FPGAs), complex programmable logic devices (CPLDs), in fixed-functionality hardware logic using circuit technology such as, for example, application specific integrated circuit (ASIC), complementary metal oxide semiconductor (CMOS) or transistor-transistor logic (TTL) technology, or any combination thereof. For example, computer program code to carry out operations shown in the method 200 may be written in any combination of one or more programming languages, including an object oriented programming language such as JAVA, SMALLTALK, C++, Python, or the like and conventional procedural programming languages, such as the “C” programming language or similar programming languages.

In step 210, i.e, step a), an image acquired by a medical scanner is received. The image may be acquired by a medical scanner selected from an x-ray imaging apparatus, an MR imaging apparatus, a CT imaging apparatus, an ultrasound imaging apparatus, and/or a PET imaging apparatus. In step 220, i.e, step b), an artefact in the image is identified. The artefact may be a motion-induced artefact, a text marker added on to the image, and/or an artefact resulting from foreign bodies external to the patient.

The identification of artefacts may according to some examples be realized by analysing the classification results during the development of a machine-learning model. For example, the machine-learning model may be applied to classify image features in the image. The classification result may be analysed to determined whether a particular class exhibits an error exceeding a threshold. If a particular class exhibits an error exceeding a threshold, determining a co-occurrence of an artefact in the image. Stated differently, if a particular class exhibits a significant error, it could be the sign of a label's manifestation co-occurring with a different feature.

In step 230, i.e, step c), the artefact is segmented to obtain a segmentation mask of a region, on which to perform inpainting. Stated differently, once the co-occurrences of an artefact has been identified, they must be segmented in order to obtain a mask of the region, on which to perform inpainting.

The segmentation of the image may according to some examples to be realized with a UNET-like structure or a deep generative machine-learned model that minimizes the distance between the source image, on which the external object is present, and the generated image, on which it is absent. In some examples, it is possible to use thresholds on CAMs in order to coarsely highlight the artefact(s) and remove them.

In step 240, i.e, step d), inpainting is performed on the region to obtain an inpainted image. In other words, after the segmentation of foreign object, the segmentation mask can be used as a marker of the location that must be inpainted.

Several inpainting techniques may be performed on the region to obtain an inpainted image including, but not limited to, inpainting with a partial differential equation and deep generative machine-learnt model, such as GANs, VAEs, and normalizing flows.

In step 250, i.e, step e), the inpainted image is incorporated into a training set for the development of the machine-learning. Optionally, one or more features may be extracted from the inpainted image using an image processing algorithm. In addition to the inpainted image, data that labels the one or more features may also be added to the training set to generate annotated ground truth data.

Optionally, the method may further comprise training the machine-learning model on the training set.

It will be appreciated that the above operation may be performed in any suitable order, e.g., consecutively, simultaneously, or a combination thereof, subject to, where applicable, a particular order being necessitated, e.g., by input/output relations.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A computer-implemented method for training a machine-learning model for classifying image features in an image acquired by a medical scanner, comprising:
receiving the image acquired by the medical scanner;
identifying an artefact in the image;
segmenting the artefact to obtain a segmentation mask of a region, on which to perform inpainting;
performing inpainting on the region to obtain an inpainted image; and
incorporating the inpainted image into a training set for the development of the machine-learning model.

2. The computer-implemented method according to claim 1, further comprising:
extracting one or more features from the inpainted image; and
incorporating data that labels the one or more features in the inpainted image into the training set.

3. The computer-implemented method according to claim 1, wherein the artefact comprises at least one of:
a motion-induced artefact in the image;
a text marker added onto the image; and
an artefact induced by an object that is imaged along with the patient.

4. The computer-implemented method according to claim 1, wherein the image comprises one or more of the following images: a chest x-ray, computed tomography, magnetic resonance, ultrasound, positron emission tomography, and a single photon emission computed tomography image.

5. The computer-implemented method according to claim 1, further comprising:
applying the machine-learning model to classify image features in the image;
analyzing a classification result to determine whether a particular class exhibits an error exceeding a threshold; and
if a particular class exhibits an error exceeding a threshold, determining a co-occurrence of the artefact in the image.

6. The computer-implemented method according to claim 1, further comprising:
segmenting the artefact based on a threshold on a class activation map;
segmenting the artefact with a UNET-like structure; or
segmenting the artefact with a deep generative machine-learnt model.

7. The computer-implemented method according to claim 1, further comprising:
performing inpainting with a deep generative machine-learnt model; or
performing inpainting with a partial differential equation.

8. The computer-implemented method according to claim 6, wherein the deep generative machine-learnt model comprises at least one of:
a generative adversarial network, GAN;
a variational autoencoder, VAE; and
normalizing flows.

9. The computer-implemented method according to claim 1, further comprising:
training the machine-learning model on the training set.

10. The computer-implemented method according to claim 1, further comprising:
displaying the inpainted image.

11. An apparatus for training a machine-learning model for classifying image features in an image acquired by a medical scanner, comprising:
an input unit configured to receive the image acquired by the medical scanner;
a processor configured to identify an artefact in the image, to segment the artefact to obtain a segmentation mask of a region on which to perform inpainting, and to perform inpainting on the region to obtain an inpainted image; and
an output configured to incorporate the inpainted image into a training set for the machine-learning model.

12. The apparatus according to claim 11, wherein the processor is further configured to extract one or more features from the inpainted image, and to incorporate data that labels the one or more features in the inpainted image into the training set.

13-15. (canceled)

16. A non-transitory computer-readable medium for storing executable instructions, which cause a method for training a machine-learning model for classifying image features in an image acquired by a medical scanner to be performed, the method comprising:
receiving the image acquired by the medical scanner;
identifying an artefact in the image;
segmenting the artefact to obtain a segmentation mask of a region on which to perform inpainting;
performing inpainting on the region to obtain an inpainted image; and
incorporating the inpainted image into a training set for the machine-learning model.
